(19) United States
(12) Patent Application Publication
(10) Pub. No.: US 2010/0063280 A1
(43) Pub. Date: Mar. 11, 2010

(54) PROCESS FOR PREPARING CCR-5 RECEPTOR ANTAGONISTS UTILIZING 4-SUBSTITUTED 1-CYCLOPROPANE-SULFONYL-PIPERIDINYL COMPOUNDS

(75) Inventors: John F. Traverse, Roselle Park, NJ (US); Shen-Chun Kuo, Union, NJ (US); Feng Liang, Monmouth Junction, NJ (US); William W. Leong, Westfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/519,729

(22) PCT Filed: Dec. 20, 2007
(86) PCT No.: PCT/US2007/026049

§ 371 Date: Nov. 2, 2009

Related U.S. Application Data
(60) Provisional application No. 60/876,888, filed on Dec. 22, 2006.

(51) Int. Cl. C07D 403/14

(52) U.S. Cl. 544/295

(57) ABSTRACT

The present invention discloses a novel process to prepare 4-substituted 1-Cyclopropane-sulfonyl-Piperidinyl compounds, which are useful intermediates for the preparation of antagonists of CCR5 receptor and therefore useful for the treatment of HIV virus infected mammals. It specifically discloses a novel process to synthesize 4-[4-[(R)-[1-[cyclopropylsulfonyl)-4-piperidinyl](3-fluorophenyl)methyl]-3(S)-methyl-1-piperazinyl]-1-[(4,6-dimethyl-5-pyrimidinyl)carbonyl]-4-methylpiperidine] compounds.

### FIELD OF THE INVENTION

This application discloses a novel process for the synthesis of the CCR5 receptor antagonist 4-[4-[(R)-[1-[cyclopropylsulfonyl)-4-piperidinyl](3-fluorophenyl)methyl]-3(S)-methyl-1-piperazinyl]-1-[(4,6-dimethyl-5-pyrimidinyl)carbonyl]-4-methylpiperidine.

### BACKGROUND OF THE INVENTION

4-[4-[(R)-[1-[cyclopropylsulfonyl)-4-piperidinyl](3-fluorophenyl)methyl]-3(S)-methyl-1-piperazinyl]-1-[(4,6-dimethyl-5-pyrimidinyl)carbonyl]-4-methylpiperidine], the compound of Formula Id having the structure shown below:

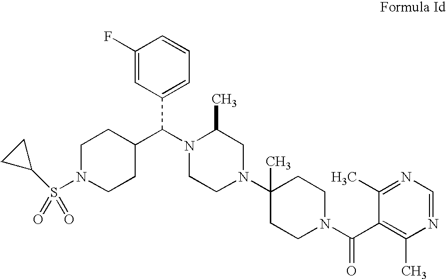

The compound of Formula I is an antagonist of the CCR5 receptor and is useful for the treatment of AIDS and related HIV infections. CCR5 receptors have also been reported to mediate cell transfer in inflammatory diseases such as arthritis, rheumatoid arthritis, atopic dermatitis, psoriasis, asthma and allergies, and inhibitors of such receptors are expected to be useful in the treatment of such diseases, and in the treatment of other inflammatory diseases or conditions such as inflammatory bowel disease, multiple sclerosis, solid organ transplant rejection and graft v. host disease. This compound is described and claimed in Example 1BF of U.S. Pat. No. 6,720,325, (the '325 patent), the entire disclosure of which is incorporated herein by reference. The '325 patent describes a synthesis of the compound of Formula Id utilizing a step-wise synthetic scheme which builds up a 4-aldehyde-substituted piperidine “left half” intermediate to the compound of Formula Id and a 4-substituted piperazine “right half” intermediate of the compound of Formula Id. The left and right half intermediates are joined in a subsequent amidation reaction in the presence of benzotriazole, providing an intermediate which undergoes further derivatization reactions to form the compound of Formula Id. An improved synthesis scheme for preparing the compound of Formula Id is described in Published International Application No. WO 2006/074270 (the '270 publication), and is illustrated below in Scheme I.

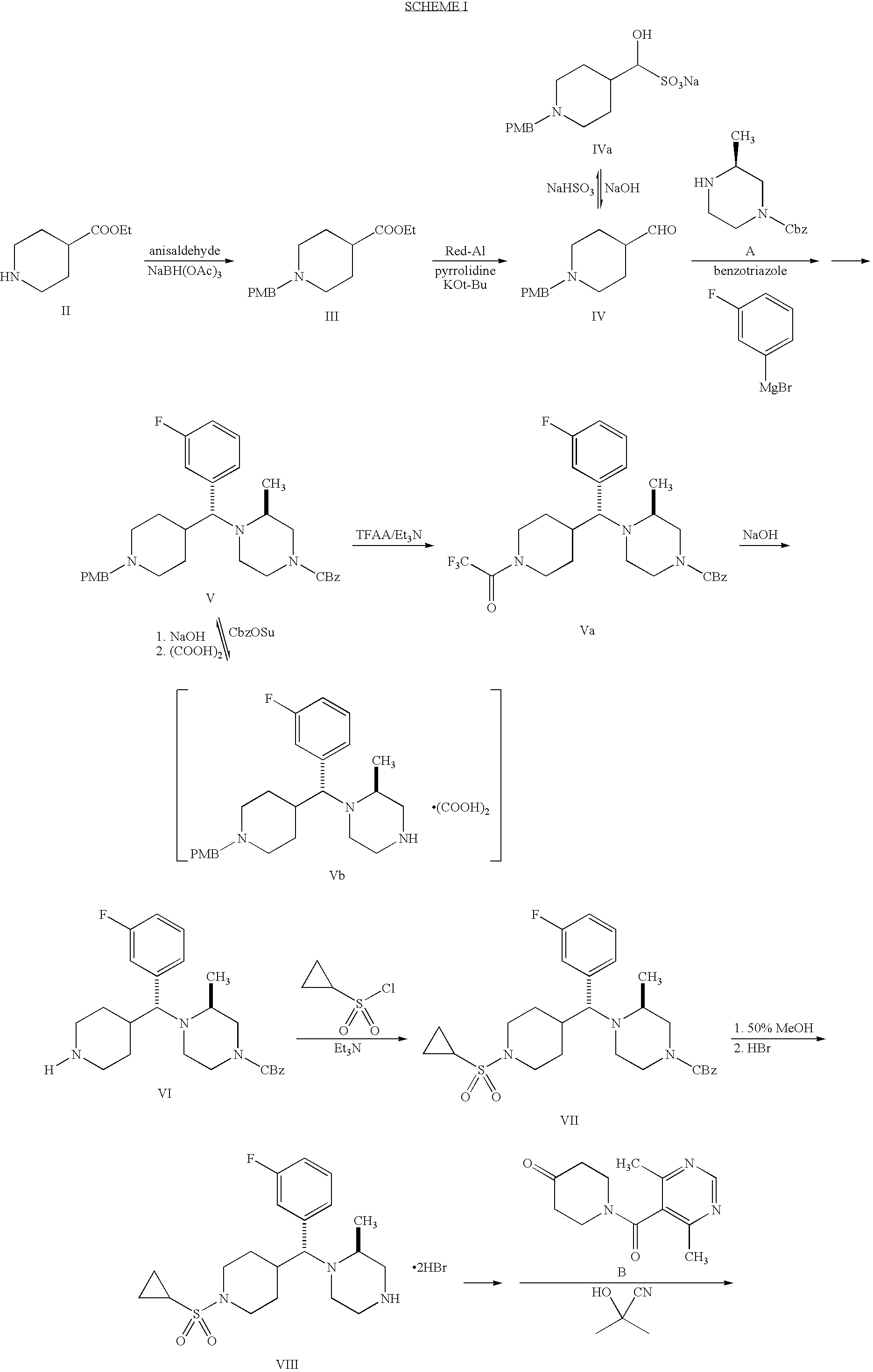

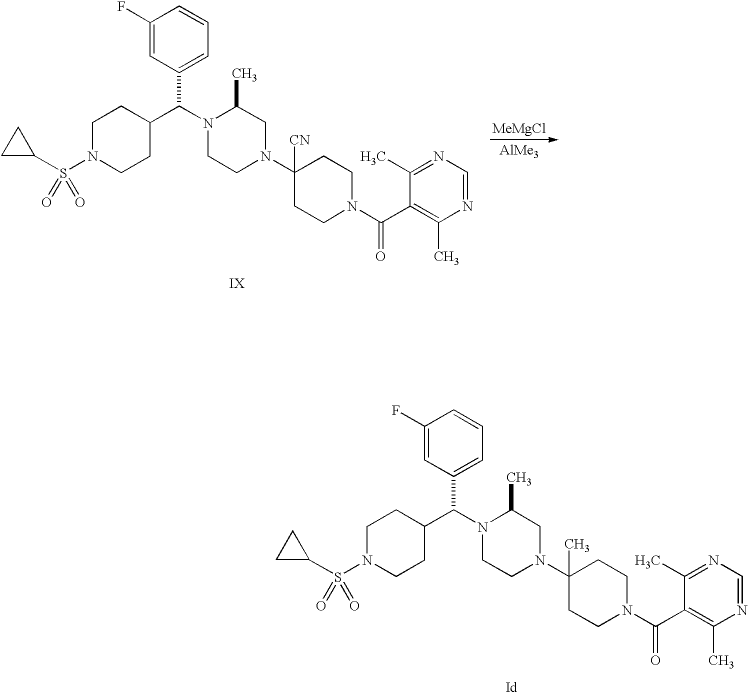

The synthetic method described in the '270 publication utilizes intermediate compound V, which has two amine groups protected, respectively, by PMB and CBZ protecting groups. The synthetic method utilizes the reactivity differences of the two protecting groups to enable further substitution of the intermediate, providing the compound of Formula Id. The disclosure of the '270 publication is incorporated by reference herein in its entirety. The above-described synthetic processes utilize numerous steps to provide the desired CCR5 receptor antagonist compound. Each of the processes described above processes proceed through intermediates having poor handling characteristics, making scale up of the processes to commercial scale problematic. Some of the steps in the above-described processes are characterized by poor yields, making cost effective production of the desired receptor antagonist problematic. In some processes, additional purification steps are required, for example, precipitating and purifying a bisulfite adduct of the compound, which further reduces the efficiency of the process from the standpoint of material utilization and processing time.

### Objectives

In view of the foregoing, what is needed is a synthetic scheme useful for preparing the CCR5 inhibitor compound of Formula Id which requires fewer steps, utilizes safer and/or more tractable materials and provides a reaction scheme affording practical scale up to a batch size suitable for commercial scale preparation. These and other objectives and/or advantages are provided by the present invention.

### SUMMARY OF THE INVENTION

In one embodiment, the present invention is a process for preparing the compound of Formula I,

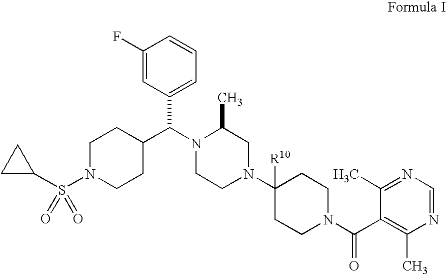

the process comprising:
(a) synthesizing the compound A4

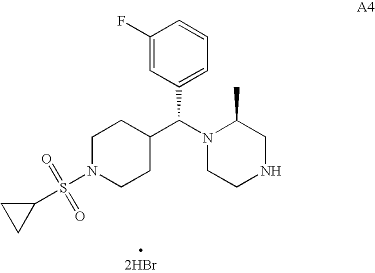

by reacting the compound A3

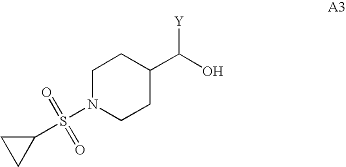

wherein “Y” is selected from: (i) —CN; (ii) —O—S(O)₂R⁴, wherein R⁴ is selected from alkyl and aryl; (iii) a halogen;
(iv) triazole

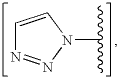

and benzotriazole

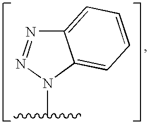

successively with: (1) 3-methylpiperidine-1-carboxylic acid benzyl ester; (2) 3-fluorophenyl magnesium bromide; and (3) HBr, in accordance with scheme 1c

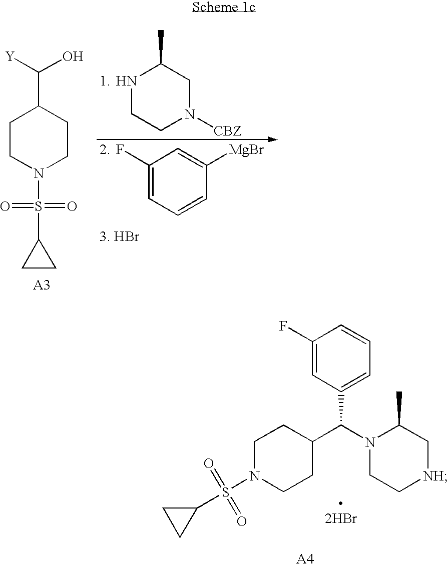

(b) coupling the free base form of intermediate compound A4 formed in Step “a” with the compound of Formula D1

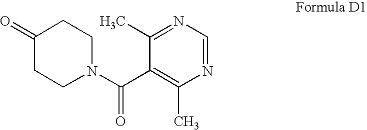

in the presence of a reactant having the form “E-G”, thereby forming the compound of Formula IX,

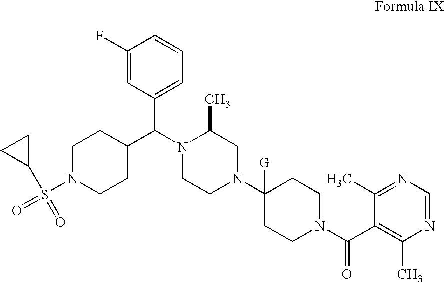

wherein, for the “E-G” reagent, “G” is selected from: (i) CN; (ii) a sulfonate ester of the Formula [—OS(O)₂—R¹], wherein R¹ is selected from an alkyl or aryl group; (iii) halogen; (iv) —C(O)—O—CX₃, wherein “X” is a halogen; and (iv) benzotriazolyl, and wherein “E” is an electrophile capable of scavenging the oxygen of the ketone carbonyl group of the compound of Formula D1 upon nucleophilic attack at the corresponding carbonyl carbon; and
(c) reacting the compound of formula IX in a suitable solvent with an organometallic reagent supplying an R¹⁰ moiety, where R¹⁰ is selected from an aliphatic and an aromatic moiety, followed by a workup to yield the compound of Formula I.

In some embodiments, preferably the compound of Formula A3 is a compound of Formula A3′

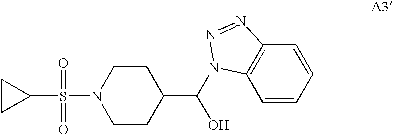

In some embodiments, preferably the organometallic reagent used in Step “c” is selected from an organometallic reagent supplying an R¹⁰ moiety selected from alkyl, for example, methyl, aryl, alkaryl, for example, benzyl, alkenyl, for example, allyl, allenyl and alkynyl, for example, propargyl. In some embodiments it is preferred to select the organometallic reagent supplying the R¹⁰ moiety from magenesium, lithium, zinc, and tin organometallic reagents, more preferably, the organometallic reagent is a magenesium organometallic reagent, more preferably, alkyl Grignard reagents. In some embodiments it is preferred to use methyl Grignard as the organometallic reagent in Step “c”, thus “R¹⁰” in the compound of Formula I is (methyl-).

In some embodiments it is preferred to select the compound E-G from cyanating agents, for example, HCN, acetone cyanohydrin; cyclohexanone cyanohydrin; a mixture of (C₂H₅)₂AlCN and Ti(OPr)₄; a mixture of acetic acid, and H₂SO₄ with NaHSO₄, KHSO₃ or Na₂S₂O₅ and a cyanide source such as NaCN or KCN; trimethylsilylcyanide; glycolonitrile; mandelonitrile; glycinonitrile; acetone amino nitrile; and dimethylaminoacetonitrile. More preferably, the E-G compound is acetone cyanohydrin.

In some embodiments, it is preferred to provide the compound of Formula A3 by the process comprising:
(a) converting a sulfonamide of Formula A1

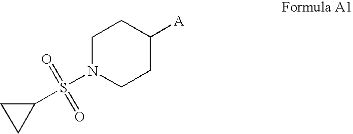

wherein substituent “A” is selected from: (i) a substituent of the Formula —C(O)—X, wherein “X” is selected from: halogen; trialkylsilane; NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety, or R² and R³ taken together form a ring; and —S—R¹ and —O—R¹, wherein R¹ is selected from hydrogen, an aliphatic moiety, including an alkyl, alkyaryl, an aromatic moiety, and a heterocyclic moiety; (ii) an alkyl-alkenyl-substituent of the formula —CHC(R²⁰)₂ wherein R²⁰ is independently selected from H, alkyl and aryl; (iii) —CN; and (iv) —CH₂OH,
   to an aldehyde compound of the Formula A2b,

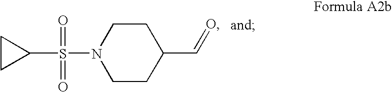

(b) reacting the aldehyde of Formula A2b with benzotriazole to form the benzotriazole-adduct of Formula A3.

In some embodiments, for example when substituent “A” in the compound of Formula A1 is CN, or contains a carbonyl carbon, for example: when “A” is C(O)—X, wherein “X” is halogen and —O—R¹, wherein R¹ is selected from hydrogen and an aliphatic, aromatic, and a heterocyclic moiety; and when “A” a substituent of the Formula —C(O)NR²R³, where R² and R³ is defined above, it is preferred to use reducing conditions to prepare the aldehyde intermediates, for example, treatment with dilsobutyl aluminum hydride (DibAlH) followed by an aqueous workup. In some embodiments, for example, when “A” in the compound of Formula A1 is an alcohol, for example CH₂—OH, it is preferred to use oxidizing conditions to prepare aldehyde intermediate. Examples of oxidizing conditions include treatment with enzymatic alcohol dehydrogenase, Dess-Martin periodate, Swern Oxidation, Moffat Oxidation, inorganic catalyst mediated oxidation, for example, oxidation using N-methylmorpholine oxide mediated by RuCl₃, and organic catalyst mediated oxidation, for example oxidation with sodium hypochlorite catalyzed by 2,2,6,6-tetramethyl-1-piperinyloxy (TEMPO).

In some embodiments wherein “A” is selected to be an alkyl-alkenyl-moiety, it is preferred to carry out oxidation with ozone followed by a suitable workup. Examples of suitable workup include reductive workup using dimethylsulfide, and dihydroxylation followed by diol cleavage.

In some embodiments, it is preferred to provide the compound of Formula A1 from the compound of Formula A1c in accordance with Scheme IIA

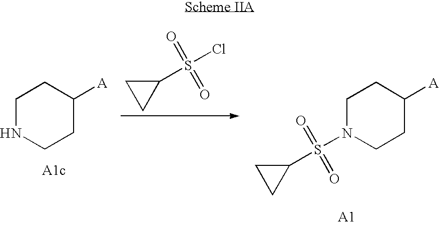

wherein substituent “A” is selected from: (i) a substituent of the Formula —C(O)—X, wherein “X” is selected from: halogen; trialkylsilane; NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety, or R² and R³ taken together form a ring; and —S—R¹ and —O—R¹, wherein R¹ is selected from hydrogen, an aliphatic moiety, including an alkyl, alkyaryl, an aromatic moiety, and a heterocyclic moiety; (ii) an alkyl-alkenyl-substituent of the formula —CHC(R²⁰)₂ wherein R²⁰ is independently selected from H, alkyl and aryl; (iii) —CN; and (iv) —CH₂OH.

In some embodiments it is preferred to select the compound of Formula A1 to be the nitrile-substituted compound of Formula A2a and convert it to an aldehyde by reaction with diisobutyl aluminum hydride (DIBAL-H), followed by an acid workup and prepare in situ therefrom the corresponding benzotriazole-adduct of Formula A3 in accordance with Scheme 1 B1

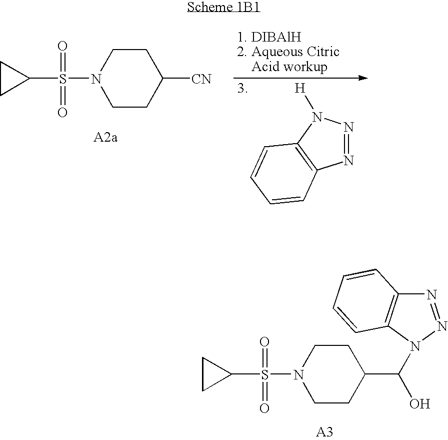

In some embodiments it is preferred to provide the compound of Formula A2a by dehydrating the corresponding sulfonamide-4-amide compound of Formula A1a, for example, by treatment with phosphorous oxychloride in accordance with Scheme 1 B2.

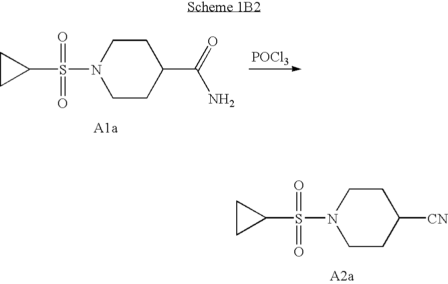

In some embodiments it is preferred to provide intermediate compound A3 in accordance with Scheme IIB,

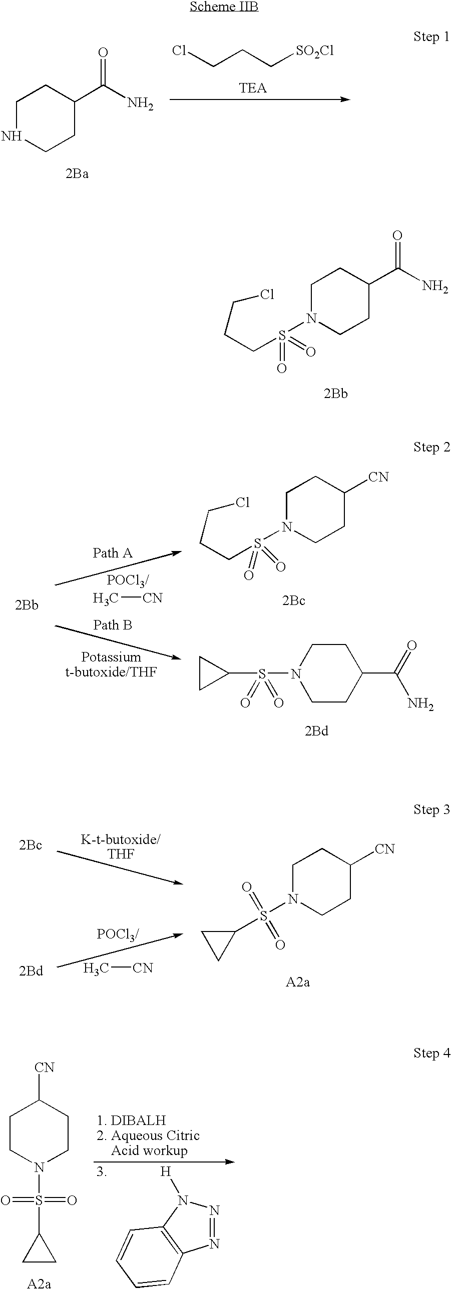

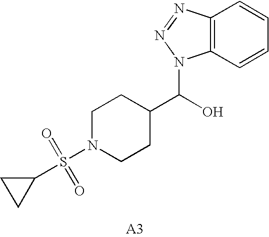

synthesis scheme IIB comprising:
(i) reacting isonipecotamide (compound 2Ba) with 3-chloropropane-sulfonyl chloride to form the adduct compound 2Bb;
(ii) following Path A to convert the amide substituent of compound 2Bb to a nitrile adduct, thereby forming compound 2Bc or alternatively following Path B, cyclizing the chloropropyl moiety of the sulfonamide substituent to form compound 2Bd;
(iii) When Path A was followed in Step 2, cyclizing the 3-chloropropyl substituent on the sulfonamide substituent of compound 2Bc to form compound A2a, and when Path B was followed in Step 2, converting the amide substituent of compound Bd to a nitrite substituent; and
(iv) reacting compound A2a sequentially with: (1) diisobutyl aluminum hydride (DibAlH) to reduce the cyano substituent to the corresponding aldehyde; (2) aqueous citric acid; and (3) benzotriazole to form benzotriazole adduct compound A3.

In some embodiments of Scheme IIB it is preferred to utilize acetonitrile (ACN) as a solvent and triethylamine (TEA) as a base in the formation of the sulfonamide in Step 1. In some embodiments utilizing Scheme IIB, it is preferred to perform conversion of the amide substituent in the compound of Formula 2Bd or 2Bb to a nitrile substituent by treating compound 2Bb or 2Bd with phosphorous oxychloride in acetonitrile. In some embodiments it is preferred to perform the nitrile conversion utilizing other dehydrating agents, for example, thionyl chloride, phosgene, phosphorous pentoxide, and oxalyl chloride. In some embodiments using alternative dehydrating agents to perform this transformation, conditions, for example, those described in Comprehensive Organic transformations, 2^{nd} ed., R. C. Larock, Wiley-VCH, NY 1999, on pages 1983 to 1985, which are incorporated herein by reference, are employed. In some embodiments utilizing Scheme IIb, it is preferred to carry out the cyclization to form the cyclopropane substituent by treating compound 2Bc with potassium tert-butoxide in tetrahydrofuran (THF) solvent.

In some embodiments it is preferred to carry out an alternative form of Scheme IIB, following in Step 2, pathway B.

In some embodiments it is preferred to prepare the compound of Formula Id,

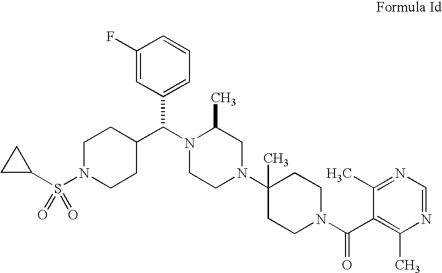

by a process comprising:
(i) reacting isonipecotamide with 3-chloropropane-sulfonyl chloride to form the adduct compound 2Bb,

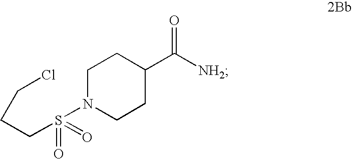

(ii) converting the amide substituent of compound 2Bb to a nitrile, thereby forming compound 2Bc,

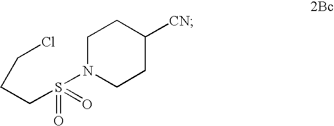

(iii) cyclizing the 3-chloropropyl substituent on the sulfonamide substituent of compound 2Bc to form compound A2a,

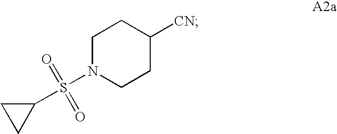

(iv) converting compound A2a to the corresponding aldehyde by reduction of the nitrile substituent with DIBAL-H followed by an acid workup and subsequently reacting the aldehyde in situ with benzotriazole to form benzotriazole adduct compound of Formula A3,

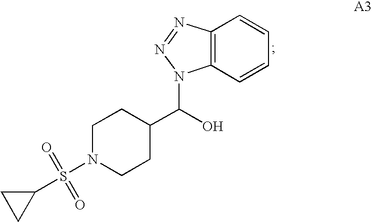

(v) synthesizing the compound A4,

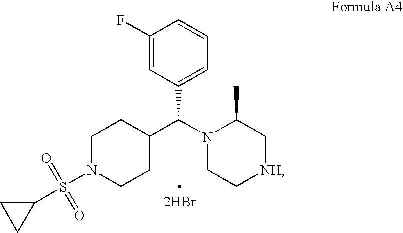

by reacting the compound A3

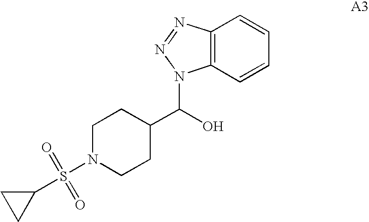

successively with: (i) 3-(S)-methylpiperazine-1-carboxylic acid benzylester; (ii) 3-fluorophenyl magnesium bromide; and (iii) HBr;
(vi) liberating the free base of compound A4 from the hydrobromide salt prepared in Step “v”;
(vii) reacting the free base liberated in Step “vi” with the compound of Formula D1,

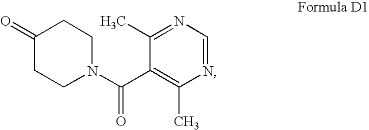

in the presence of a moiety of the Formula “E-G”, where “E” is an electrophile capable of scavenging the oxygen of the ketone carbonyl group of the compound of Formula D1 upon nucleophilic attack at the corresponding carbonyl carbon and “G” is a leaving group selected from the group consisting of CN, halogen, —OSO₂—R¹ (wherein R¹ is selected from an alkyl or aryl group), —C(O)OCX₃ (wherein “X” is a halogen), and benzotriazolyl, to form the compound of Formula IX,

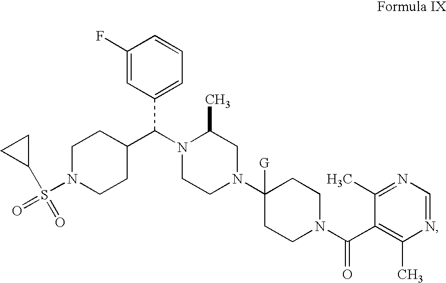

wherein “G” is as defined above for the E-G reagent selected; and
(viii) reacting the compound of Formula IX with a methyl Grignard reagent to yield the compound of Formula Id.

In some embodiments the present invention provides the following compounds:

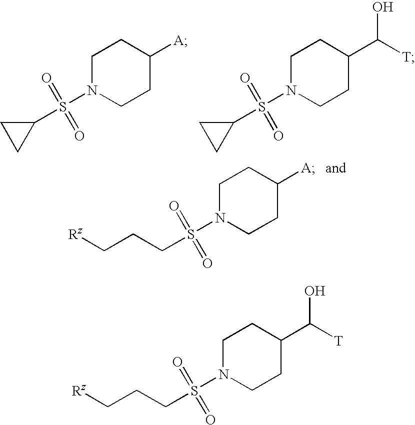

wherein, “A” is as defined above, T is selected from: (i) CN; (ii) a sulfonate ester of the Formula [—OS(O)₂—R¹¹], wherein R¹¹ is selected from an alkyl or aryl group; (iii) halogen; (iv) —C(O)—O—CX₃, wherein “X” is a halogen; (v) triazole; and (vi) benzotriazole, and R^{z} is selected from: a halogen and or O—R¹³ where R¹³ is selected from —C(O)R¹⁴, —C(O)OR¹⁴, or S(O)₂R¹⁴, where R¹⁴ is H, an aliphatic moiety and an aromatic moiety.

### DETAILED DESCRIPTION OF THE INVENTION

As mentioned above, and described in published international application no. 2006/074270, the compound of Formula Id is a CCR5 receptor antagonist and is useful in the treatment of AIDS and related HIV infections and may be useful in the treatment of other diseases, for example, inflammatory diseases. Except where stated otherwise, the following definitions apply throughout the present specification and claims. These definitions apply regardless of whether a term is used by itself or in combination with other terms.

“Acyl” means an H—C(O)—, alkyl-C(O)—, cycloalkyl-C(O)—, or aryl-C(O)— and the like. The bond to the parent moiety is through the carbonyl.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that one or more hydrogen atoms on an alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“alkyl-alkenyl-” means one or more aliphatic hydrocarbon groups of from about 1 to about 20 carbon atoms bonded to a double bonded hydrocarbon moiety, for example, —(H)C═C(R₂), wherein R is selected independently for each occurrence from any of the substituents herein mentioned.

“Alkylsulfonate” means an alkyl-S(O₂)—O— group in which the alkyl group is as previously described. Preferred groups are those in which the alkyl group is lower alkyl. The bond to the parent moiety is through the oxygen.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Cbz” means carbobenzyloxy.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms.

The terms “Halide”, “Halo” and “Halogen” mean fluoro, chloro, bromo or iodo moieties.

The terms “Heterocycyl” and “Heterocyclic” refer to a non-aromatic saturated monocyclic or multicyclic ring system comprising about 3 to about 10 ring atoms, preferably about 4 to about 7 ring atoms, in which one or more of the atoms in the ring system is an element other than carbon, for example, nitrogen, oxygen or sulfur, alone or in combination, with the proviso that no adjacent oxygen and/or sulfur atoms are present in the ring system. Preferred heterocyclyl groups contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist in a protected form, for example, an —N(Boc), —N(cbz), —N(Tos) group. Such protections are also considered part of this invention.

The term “heterocyclic” as used herein, also includes heteroaryl, which, as used herein, is a mono-, bicyclo, or polycyclic, chemically feasible ring system containing one or more aromatic rings having in at least one aromatic ring at least 1, up to about 4 nitrogen, oxygen or sulfur atoms. Typically, a heteroaryl group represents a cyclic group of five or six atoms, or a bicyclic group of nine or ten atoms, or a polyfused ring system with each ring containing from about 4 to about 6 atoms, at least one of which is carbon, and having at least one oxygen, sulfur or nitrogen atom interrupting a carbocyclic ring having a sufficient number of pi (π) electrons to provide aromatic character. Representative heteroaryl (heteroaromatic) groups are pyridinyl, pyrimidinyl, pyrazinyl, pyridazinyl, furanyl, benzofuranyl, thienyl, benzothienyl, thiazolyl, thiadiazolyl, imidazolyl, pyrazolyl, triazolyl, tetrazolyl, isothiazolyl, benzothiazolyl, benzoxazolyl, oxazolyl, pyrrolyl, isoxazolyl, 1,3,5-triazinyl and indolyl groups. The heteroaryl group can be joined to the rest of the molecule through a bond at any substitutable carbon or nitrogen.

“PMB” means p-methoxybenzyl.

“Triflate” means trifluoromethanesulfonyl.

The term “derivative thereof” used in connection with a compound means a structurally related compound which is obtainable by common chemical transformation of one or more functional groups existing or derivable from groups existing on the original molecule.

The present invention is an improved process for preparing the CCR5 receptor antagonist compound of Formula I, preferably wherein R¹⁰ is CH₃, and is therefore the compound of Formula Id.

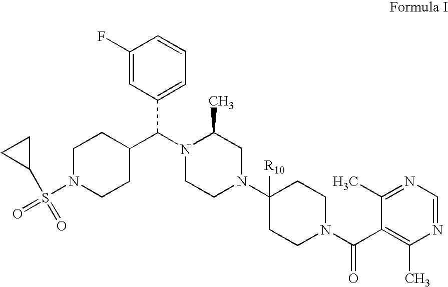

In one embodiment, the present invention is an improved process for preparing the intermediate compound of Formula A4,

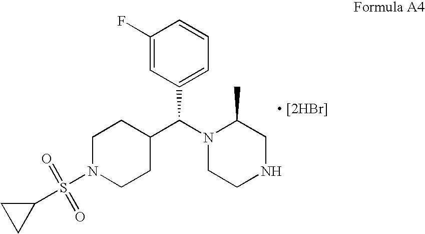

from which, in accordance with Scheme III, the compound of Formula I is prepared.

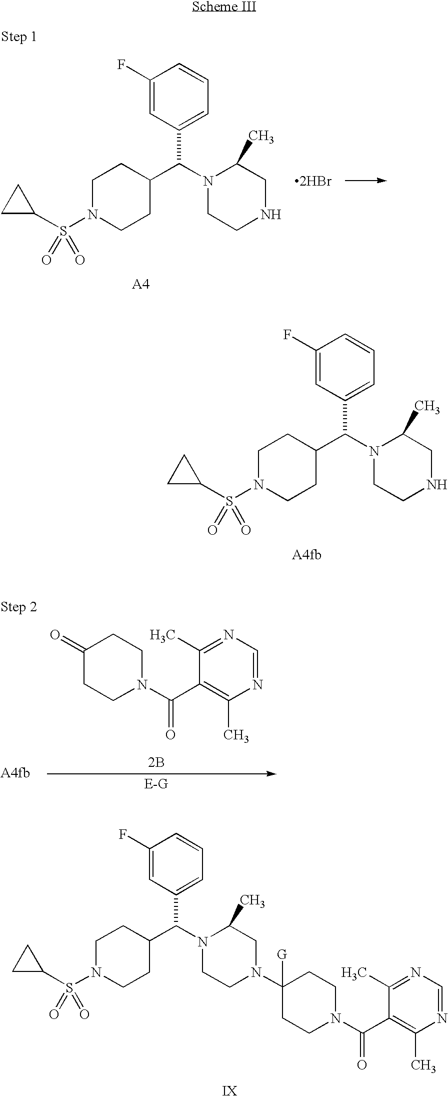

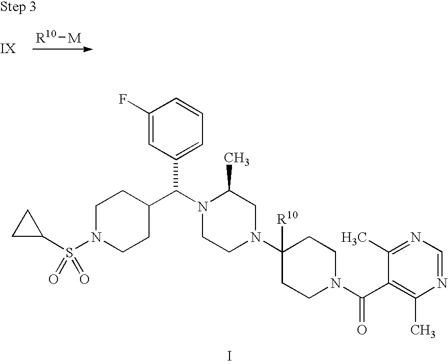

wherein R¹⁰-M is an organometallic reagent supplying R¹⁰, preferably the organometallic reagent is methyl Grignard and R¹⁰ is methyl. The process of Scheme III for preparing the compound of Formula I is described in published international application No. WO 2006/074270 (herein, “The '270 publication”), the disclosure of which is incorporated herein by reference. Accordingly, Scheme III shows that the compound of Formula I is prepared by converting the compound of Formula A4 into a free base form (shown in Scheme III as the compound of Formula A4fb), by treating the compound with a base, for example, sodium carbonate. Once obtained the free base compound A4fb is reacted with piperidin-4-one compound 2B (step 2 of Scheme III) in the presence of a reactant having a facile leaving group (G) and an electrophilic group (E) capable of scavenging the oxygen of the piperidin-4-one moiety during the addition reaction. As described in the '270 publication, examples of compounds having such an E-G structure include cyanating agents, for example, HCN, acetone cyanohydrin; cyclohexanone cyanohydrin; a mixture of (C₂H₅)₂AlCN and Ti(OPr)₄, a mixture of acetic acid, H₂SO₄; NaHSO₄, KHSO₃ or Na₂S₂O₅ and a cyanide source such as NaCN or KCN; trimethylsilylcyanide; glycolonitrile; mandelonitrile; glycinonitrile; acetone amino nitrile; and dimethylaminoacetonitrile. Most preferably, the E-G reagent is 2-hydroxy-2-methyl-propionitrile,

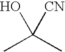

(acetone cyanohydrin).

Without wishing to be bound by theory, it is believed that providing an E-G compound to the reaction mixture promotes addition of the nitrogen group of compound A4 to carbon 4 of the piperidine ring. It is further believed that the E-G reagent concomitantly supplies a facile leaving group substituent which, in the course of the reaction, is transferrred to carbon 4 of the piperidine ring. Thus, the E-G reagent provides a substituent to the intermediate compound of Formula IX which is easily replaced in a subsequent step by an carbanionic R¹⁰ group via reaction of the intermediate of Formula IX with an organometallic ragent supplying R¹⁰. Preferably the organometallic reagent is a methyl Grignard reagent, supplying —CH₃ forming the compound of Formula Id wherein a R¹⁰ is methyl.

In one embodiment, the present invention provides compound A4 in accordance with Scheme 1c, by reacting the compound of Formula A3 with 3-(S)-methypiperazine-1-carboxylic acid benzyl ester, having a CBZ protecting group on the nitrogen of ring position no. 1, replacing the benzotriazole moiety on A3. The product of this reaction is subsequently reacted in situ with a 3-fluoro-benzyl-Grignard reagent, for example, 3-fluoro-magnesium bromide, followed by treatment of the reaction mixture with HBr to remove the CBZ protecting group and precipitate the product (the compound of the Formula A4) as the HBr salt.

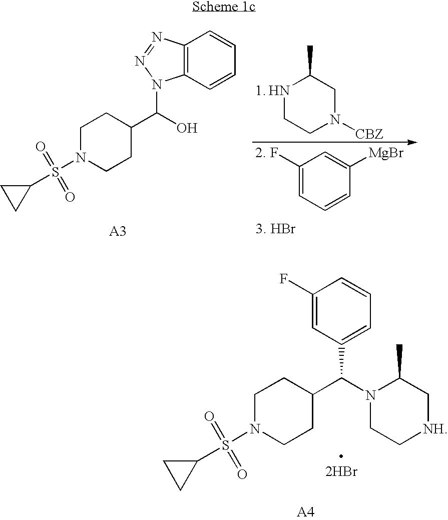

The reaction shown in Scheme 1c is preferably carried out in accordance with published conditions, for example, those described in published international application no. WO 03/084942, which also describe preparation of 3-methyl-piperazine-1-CBZ. Preferred conditions for carrying out the reaction include carrying out Step 1 of the reaction in refluxing toluene catalyzed by para-toluenesulfonic acid, with azeotropic distillation to remove water. Step 2 is preferentially carried out with the reaction mixture maintained at a temperature of from about 0° C. to about 5° C. Phase 3 is preferentially carried out by addition of aqueous HBr to the reaction mixture, agitation for 10 minutes with subsequent removal of the aqueous layer, followed by heating for 3 hours to about 70° C. Subsequent cooling and the addition of isopropanol is preferred to precipitate the HBr salt of A4.

Accordingly, the method of the present invention provides compound A4 directly as the sulfonamide from an isolated benzotriazole adduct (the compound of Formula A3) without the need to provide a precursor having two different protecting groups, which are removed sequentially to permit addition of the sulfonamide group to the desired nitrogen and leave the other nitrogen available for further reactions after the sulfonamide group is put in place.

As shown in Scheme 1c, the no. 1 nitrogen of the piperazine moiety is preferably protected by Cbz, (carbobenzyloxy moiety), an acid-labile nitrogen protecting group. Other acid labile nitrogen protecting groups may alternatively be used, for example, CZ₃CO (where Z is a halogen), 2-trimethylsilylethyl carbamate, 1-methyl-1-phenylethyl carbamate, t-butyl carbamate, cyclobutyl carbamate, 1-methylcyclobutyl carbamate, adamantyl carbamate, vinyl carbamate, allyl carbamate, cinnamyl carbamate, 8-quinolyl carbamate, 4,5-diphenyl-3-oxazolin-2-one, benzyl carbamate, 9-anthrylmethyl carbamate, diphenylmethyl carbamate, S-benzylcarbamate, methyl carbamate, ethyl carbamate, diphenylphosphinyl, benzenesulfenyl, RCO (where R is C₁₋₆alkyl), benzoyl and other common acyl groups. Alternatively, it will be appreciated that the N-1 nitrogen of the piperazine moiety can be protected with base-labile protecting groups, for example, 9-fluorenylmethyl carbamate (FMOC), which are later removed by treatment with a base rather than an acid. It will be appreciated that other protecting groups may alternatively be used in the present process, for example, those described in Protecting Groups in Organic Synthesis, 3^{rd} ed., T. Green, W. Theodora, and P. G. M. Wuts, John Wiley & Sons, NY 1999, pp 504 to 615, which is incorporated herein by reference in its entirety. It will also be appreciated that deprotection of the nitrogen moiety initially protected by the protecting group can be carried out using other methods than treatment with HBr, for example, treating with other protic acids, for example HCl, HI, and H₂SO₄, treating with Lewis acids, for example AlX₃, and BX₃, wherein “X” is a halogen, as well as by hydrogenation, for example, treatment of the compound with H₂ in the presence of a hydrogenation catalyst, for example, palladium on carbon.

In one embodiment of the present invention, the compound of Formula A3 is provided by preparing an aldehyde intermediate (A2b) from the compound of Formula A1 in accordance with Scheme 1b2. The compound of Formula A1 is either oxidized or reduced, depending upon the “A” substituent present, to provide the intermediate aldehyde (compound A2b) which is in turn reacted with benzotriazole to provide intermediate A3. Thus A3 is prepared in accordance with Scheme 1b2.

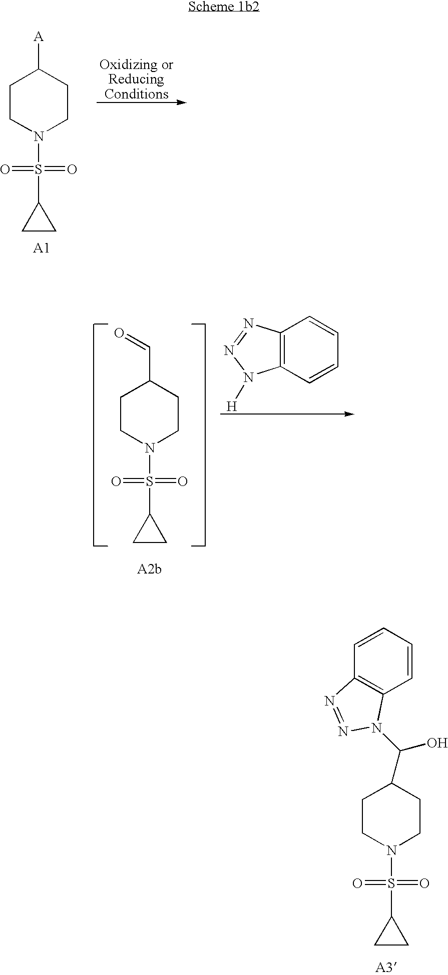

wherein substituent “A” is selected from: (i) a substituent of the Formula —C(O)—X, wherein “X” is selected from: halogen; trialkylsilane; NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety, or R² and R³ taken together form a ring; and —S—R¹ and —O—R¹, wherein R¹ is selected from hydrogen, an aliphatic moiety, including an alkyl, alkyaryl, an aromatic moiety, and a heterocyclic moiety; (ii) an alkyl-alkenyl substituent of the formula —CHC(R²⁰)₂ wherein R²⁰ is independently selected from H, alkyl and aryl; (iii) —CN; and (iv) —CH₂OH. Preferably, the “A” substituent is selected from: (i) a substituent of the Formula —C(O)—X, wherein “X” is selected from halogen, trialkylsilane, and —O—R¹, wherein R¹ is selected from hydrogen and an aliphatic, aromatic, or heterocyclic moiety; (ii) a substituent of the Formula —C(O)NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety; (iii) —CN; and (iv) —CH₂OH.

For preparation of the intermediate aldehyde of Formula A2b, reducing conditions are selected when the “A” substituent is —CN or contains a carbonyl group bonded to the piperazine ring, for example, when “A” is C(O)—X. Preferably, reducing reagents are selected from hydride reducing agents, for example, diisobytyl aluminum hydride (DIBAL-H), sodium bis(2-methoxyethoxy)aluminum hydride (RED-Al®), and lithium aluminum hydride. When “A” is an acid halide, reduction can be carried out using hydrogen in the presence of a noble metal catalyst, for example Pd. When “A” is, for example, an alcohol, for example, CH₂OH. In some embodiments, for example, when “A” in the compound of Formula A1 is an alcohol, for example CH₂—OH, it is preferred to use oxidizing conditions to prepare aldehyde intermediate. Examples of oxidizing conditions include treatment with enzymatic alcohol dehydrogenase, Dess-Martin periodate, Swern Oxidation, Moffat Oxidation, inorganic catalyst mediated oxidation, for example, oxidation using N-methylmorpholine oxide mediated by RuCl₃, and organic catalyst mediated oxidation, for example oxidation with sodium hypochlorite catalyzed by 2,2,6,6-tetramethyl-1-piperinyloxy (TEMPO).

In some embodiments wherein “A” is selected to be an -alkenyl moiety, it is preferred to carry out oxidation with ozone followed by a suitable workup. Examples of suitable workup include reductive workup using dimethylsulfide, and dihydroxylation followed by diol cleavage.

Well known literature procedures for oxidation or reduction of the types of substituents appearing on compound A2a to an aldehyde functional group are found in: Acid to aldehyde: “Comprehensive Organic Synthesis” Eds. B. M. Trost and I. Fleming, Pergamon, Oxford, (1991), Vol 8, parts 1.11 and 1.12, pp 259-306; Ester to aldehyde: Feldman, K. S. et al. J. Am. Chem. Soc. 1994 116, 9019-9026; Thioester to aldehyde: Ho, P. T.; Ngy, K-y J. Org. Chem. 1993; 58, 2313-2316; Amide to aldehyde: Hagihara, M.; Schreiber, S. L. J. Am. Chem. Soc, 1992, 114, 6570-6571; Nitrile to aldehyde: Guilard, G. et al. J. Am. Chem. Soc 1992, 114, 9877-9889; Acyl silane to aldehyde: Cirillo, P. F.; Panek, J. J. Tetranderon Lett. 1991, 32, 457-460; Acid halide to aldehyde: Chen, C-y. et al. J. Org. Chem. 1994, 59, 3738-3741; Alcohol to aldehyde: Enzyme: Yamazaki, Y.; Hosono, K. Tetranderon Lett 1988, 29, 5769-5770; Alcohol to aldehyde: Chemical: Leanna, M. R.; Sowin, T. J..; Morton, H. E. Tetranderon Lett 1992, 33, 5029-5032; Alkene to aldehyde: Ozone: March J. Advanced Organic Chemistry: Reactions, Mechanisms, and Structure, 4^{th} Ed., John Wiley and Sons, New York, 1992, pp 1177 and following; Dihydroxylation and diol cleavage: ibid at p 822 and following, p 1174 and following, all of which are incorporated by reference herein in their entirety.

In one embodiment of the present invention, it is preferred for the compound of the structure of A1 to be the sulfonamide amide compound A1a (a sulfonamide with a primary amide substituent on piperidine ring carbon no. 4), it is preferred to provide intermediate compound A3 in accordance with Scheme 1b1.

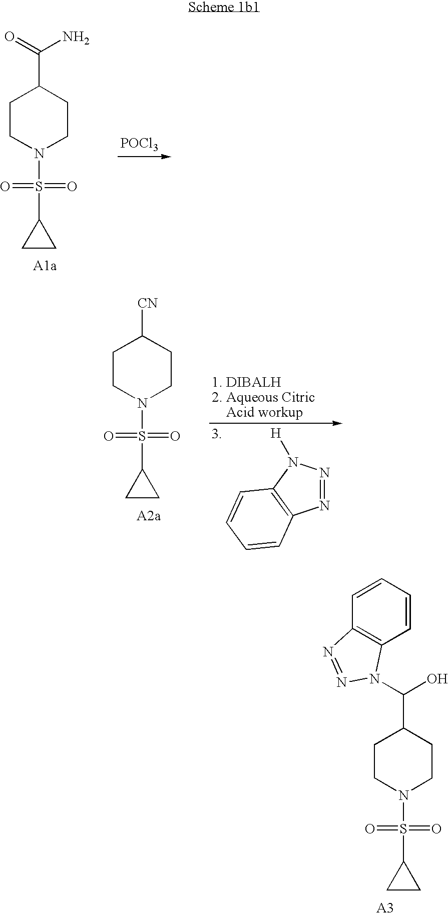

wherein the primary amide substituent of A1a is dehydrated by treatment with POCl₃ to the corresponding nitrite compound (A2a). Preferably, the dehydration is carried out in an acetonitrile solvent. In this embodiment, preferably the nitrile compound A2a is subsequently reduced to the corresponding aldehyde using diisobutyl aluminum hydride in toluene with an aqueous citric acid workup. This aldehyde intermediate is subsequently reacted, as described above, with benzotriazole to yield intermediate compound A3. It will be appreciated that conversion of the amide group of the compound of Formula A1a to a nitrile group can also be carried out using other dehydrating agents, for example, thionyl chloride, phosgene, phosphorous pentoxide, and oxalyl chloride. In some embodiments using alternative dehydrating agents to perform this transformation, known conditions are employed, for example, those described in Comprehensive Organic transformations, 2^{nd} ed., R. C. Larock, Wiley-VCH, NY 1999, on pages 1983 to 1985, which are incorporated herein by reference.

In one embodiment of Scheme 1b1, the reaction is preferably carried out by charging a toluene solution of DIBAL-H to a solution of A1a in tetrahydrofuran, (THF) at a temperature between −15° C. and 0° C. Following addition of DIBAL-H, the reaction mixture is agitated for about two hours with the temperature of the reaction mixture maintained at about 20° C. The reaction mixture is quenched with a solution of aqueous citric acid, generally by stirring the reaction mixture with the solution, for example, for a period of one hour. The organic (toluene/THF) and aqueous layers of the mixture are then separated, and the toluene/THF solution of compound A2a is retained for subsequent treatment with benzotriazole. The third step is carried out by adding a THF solution of benzotriazole the solution of aldehyde compound A2a provided by the DIBAL-H reduction. The THF is distilled off and the reaction mixture is refluxed (refluxing temperature approximately 110° C.). After completing the reaction, the reaction mixture is cooled to a temperature of from about 20° C. to about 30° C., during which benzotriazole adduct A3 precipitates out of solution. Adduct A3 is then filtered, washed with toluene, and dried.

Optionally, the intermediate A2a aldehyde compound can be isolated by removal of the solvent before forming benzotriazole adduct A3, yielding a waxy, low melting solid. The A2a compound is then stored and handled under an inert atmosphere until it is used to prepare the benzotriazole adduct to prevent oxidation.

In some embodiments of the present invention the intermediate compound of Formula A3 is a compound of Formula A3′, and it is preferred to prepare the intermediate compound of Formula A3′ in accordance with Scheme IIB,

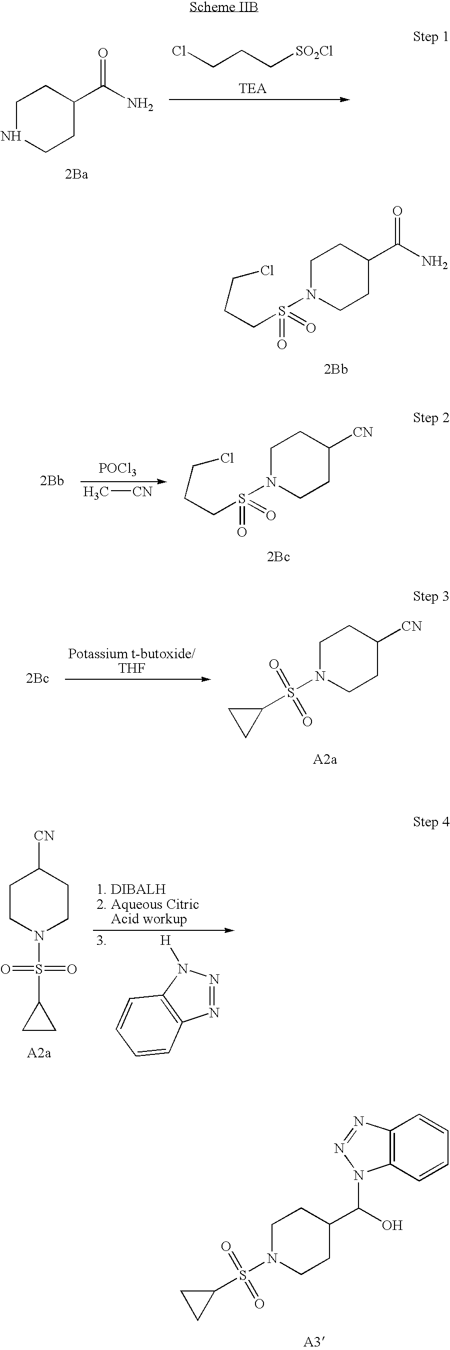

Accordingly,synthesis scheme IIB comprises:
(i) reacting isonipecotamide (compound 2Ba) with 3-chloropropane-sulfonyl chloride to form the adduct compound 2Bb;
(ii) converting the amide substituent of compound 2Bb to a nitrile substituent, thereby forming compound 2Bc;
(iii) cyclizing the 3-chloropropyl substituent on the sulfonamide substituent of compound 2Bc to form compound A2a; and
(iv) reacting compound A2a with benzotriazole to form benzotriazole adduct compound A3′.

Alternatively, starting with compound 2Bb, steps 2 and 3 can be reversed, treating the compound of Formula 2Bb with a metal alkoxide to cyclize the chloropropyl substituent on the sulfonamide moiety, yielding the cyclopropylsulfonamide amide compound of Formula 2Bd,

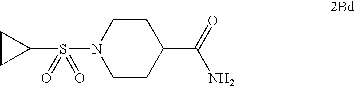

and then treating the compound of Formula 2Bd with phosphorous trichloride to convert the amide functional group to a nitrile, providing the compound of Formula A2a. The conversion of the amide group in the compound of Formula 2Bd can alternatively be accomplished with other dehydrating agents, for example, but not limited to, thionyl chloride, phosgene, phosphorous pentoxide, and oxalyl chloride. In some embodiments using alternative dehydrating agents to perform this transformation, known conditions are employed, for example, those described in Comprehensive Organic transformations, 2^{nd} ed., R. C. Larock, Wiley-VCH, NY 1999, on pages 1983 to 1985, which are incorporated herein by reference.

Variations on Scheme IIB can also include utilizing differently functional piperidine compounds in place of 2Ba for use in either the alternative or non-alternative Scheme IIB processes. Thus, for example, compound 2Ba-1 can be prepared according to Scheme IIB-1:

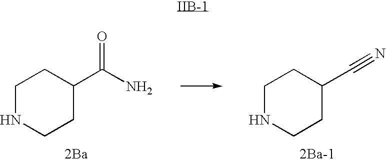

by converting the primary amide functional group to a nitrile in accordance with the above-described processes or those described in Comprehensive Organic Transformations, 2^{nd} ed., Richard Larock, Wiley-VCH, NY 1999, pp 1983 to 1985, which is incorporated herein by reference. When used in Scheme IIB, Step 1, in place of 2Ba, it provides the compound of Formula 2Bc directly. When used in the alternative embodiment of Scheme IIB, it provides the compound of Formula A2a directly in Step 1. When used as compound A1c in the Scheme of Formula IIA (below) it provides the cyclypropylsulfonamidenitrile compound of Formula A2a directly also.

By providing a method of cyclizing the cyclopropyl ring after forming the sulfonamide compound of Formula 2Bb (as shown in Scheme IIB), the need to prepare cyclopropylsulfonyl chloride is obviated. In carrying out Scheme IIB it is preferred to employ acetonitrile as a solvent and triethylamine (TEA) as a base which catalyzes the sulfonamide formation in Step (i). In some embodiments utilizing Scheme IIB, it is preferred to perform Step (ii), conversion of the amide substituent to a nitrile substituent by treating compound 2Bb with phosphorous oxychloride in acetonitrile. In some embodiments utilizing Scheme IIb, it is preferred to carry out cyclopropanation Step (iii) by treating compound 2Bc with potassium tert-butoxide in tetrahydrofuran (THF) solvent. It will be appreciated that other known base compounds having similar or greater proton affinity can be employed to cyclize the chloropropyl moiety present in either the compound of Formula 2Bb or in the compound of Formula 2Bc.

As discussed above, in some embodiments of the present invention the intermediate compound of Formula A3 is alternatively prepared from the sulfonamide compound of Formula A1, wherein the substituent “A” is selected from: (i) a substituent of the Formula —C(O)—X, wherein “X” is selected from: halogen; trialkylsilane; NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety, or R² and R³ taken together form a ring; and —S—R¹ and —O—R¹, wherein R¹ is selected from hydrogen, an aliphatic moiety, including an alkyl, alkyaryl, an aromatic moiety, and a heterocyclic moiety; (ii) an alkyl-alkenyl-substituent of the formula —CHC(R²⁰)₂ wherein R²⁰ is independently selected from H, alkyl and aryl; (iii) —CN; and (iv) —CH₂OH, where the compound of Formula A1 is prepared in accordance with synthesis Scheme IIA.

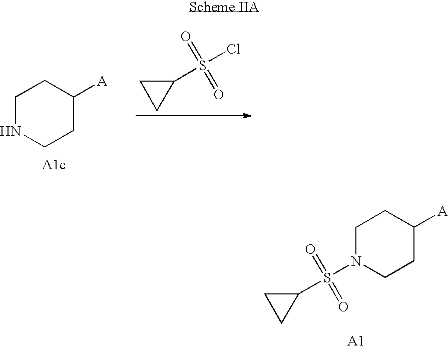

Where substituent “A” is —CH₂OH, —CONH₂, —C(O)OEt, and —C(O)OH, the compound A1c is a commercially available material. It will be appreciated that the remaining of the above listed substituents can be prepared from one or more of these commercially available materials. Preparation of cyclopropanesulfonyl chloride is known, for example, the procedure described in Organic Sulfur Mechanisms. 36. Cyclopropanesulfonyl chloride: its mechanisms of hydrolysis and reactions with tertiary amines in organic media, King, James F.; Lam, Joe Y. L.; Ferrazzi, Gabriele. Dep. Chem., Univ. West. Ontario, London, ON, Can. Journal of Organic Chemistry (1993), 58(5), 1128-35.

### Examples

The following solvents and reagents may be referred to by their abbreviations in parenthesis:
sodium bis(trimethylsilyl)amide: NaHMDS
triethyl amine: TEA
trifluoro acetic acid: TFA
tertiary-butoxycarbonyl: t-BOC
tetrahydrofuran: THF
lithium bis(trimethylsilyl)amide: LiHMDS
Methyl tent-butyl ether: MTBE
DIBAL-H: Diisobutyl Aluminum Hydride
Dimethyl Sulfoxide d⁶: DMSO
Deuterated chloroform: CDCl₃
Deuterium oxide: D₂O
mole: mol.
millimole: mmol
Hertz: Hz
Chemical Shift (NMR): δ
singlet=s
doublet=d
triplet=t
broad=b
Melting Point=MP

### Example 1

### Preparation of Sulfonamide-amide A1a

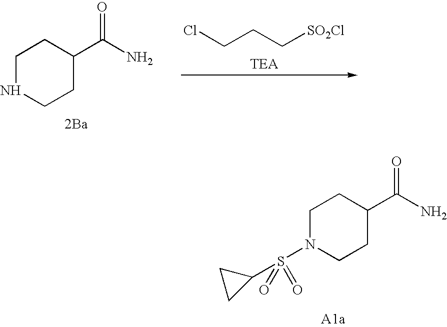

With reference to Scheme IIB, above, wherein Path B is followed in Step 2, compound 2Ba (isonipectoamide, article of commerce, used as received) (36.5 g, 285 mmol), acetonitrile (150 mL), and saturated aqueous potassium carbonate solution (150 mL) were added to a 1 liter jacketed vessel. To this mixture was charged 3-chloropropane sulfonyl chloride (40.0 g, 285 mmol), keeping the temperature between 15° C. and 25° C. The reaction was then stirred for about 15 minutes. MTBE (500 mL) was then charged, and the resulting precipitated product A1a was filtered, and washed with MTBE (3×100 mL). The solids were dried under vacuum oven at 20 to 30° C. to yield 51.2 g (77.3%) of product Ala as a white solid.

¹H NMR (DMSO, 400 MHz): δ 7.31 (1H, s), 6.86 (1H, s), 3.58 (2H, ddd, J=12.0, 3.2, 3.2 Hz), 2.81 (2H, ddd, J=12.0, 12.0, 2.4 Hz), 2.57 (1H, m), 2.21 (1H, dddd, J=10.8, 10.8, 3.2, 3.2 Hz), 1.79 (2H, dddd, J=13.2, 2.8, 2.8, 2.8 Hz), 1.53 (2H, dddd, J=13.6, 13.6, 13.6, 4.0Hz), 0.99-0.88 (4H, m). MP: 151° C.

### Example 2

### Preparation of Sulfonamide-nitrile A2a from A1a

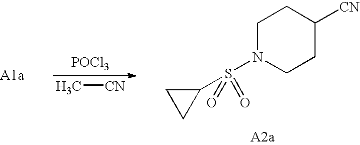

Compound A1a (50.0g, 215 mmol) and acetonitrile (200 mL) were charged to a 1 liter jacketed flask and were heated to 55 to 60° C. Phosphorous oxychloride (POCl₃, 50.1 g, 323 mmol) was added, keeping the temperature at about 60° C. The reaction was kept at 60° C. for about 6 hours, at which time toluene (250 mL) was added. The reaction was allowed to cool to 25° C. and was slowly quenched with saturated aqueous potassium bicarbonate (350 mL), keeping the temperature below 30° C. The layers were separated, and the aqueous layer was extracted with toluene (200 mL). The organic layers were combined, washed with brine (75 mL) and dried over anhydrous magnesium sulfate. The organic layer was then filtered, and the solvent was removed under reduced pressure to yield 42.1 g (91.3%) of compound A2a as an off-white solid.

¹H NMR (CDCl₃, 400 MHz): δ 3.44-3.42 (4H, m), 2.89 (1H, tt, J=6.0, 4.8 Hz), 2.27 (1H, tt, J=8.0, 4.8 Hz), 2.05-1.97 (4H, m), 1.19 (2H, m), 1.03 (2H, m). MP: 91.9° C.

### Example 3

### Preparation of Open-Chain Sulfonamide-amide 2Bb

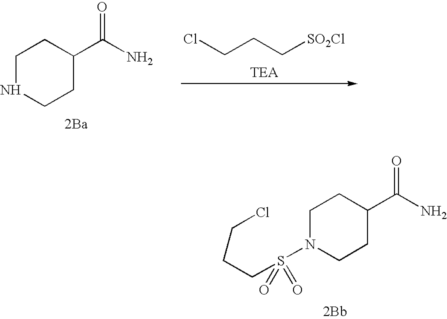

With reference to Scheme IIB (primary Scheme), Step 1, compound 2Ba (isonipectoamide) (54.7 g, 424 mmol) acetonitrile (750 mL), and triethylamine (47.2 g, 466 mmol) were charged to a 2 liter jacketed flask and agitated at about 20° C. The flask was charged 3-chloropropanesulfonyl chloride (75.0 g, 424 mmol) dissolved in acetonitrile (120 mL) through an addition funnel over one hour, keeping the temperature of the reaction mixture at about 20° C., and stirred for four to five hours after addition. To the agitating reaction mixture were added 10% aqueous citric acid (500 mL) and ethyl acetate (500 mL). The layers were allowed to split, and the bottom, aqueous layer was extracted with ethyl acetate (300 mL). The organic layers were combined and washed with 10% aqueous citric acid (400 mL) and brine (200 mL). The batch was concentrated under reduced pressure, and the resulting solids were washed with MTBE to yield 53.0 g (46.5%)compound 2Bb as an off-white solid.

¹H NMR (DMSO, 400 MHz): δ 7.32 (1H, s), 6.86 (1H, s), 3.74 (2H, dd, J=6.4, 6.4 Hz), 3.58 (2H, m), 3.14 (2H, m), 2.80 (2H, ddd, J=12.0, 12.0, 2.4Hz), 2.20 (1H, dddd, J=11.4, 11.4, 3.8, 3.8 Hz), 2.09 (2H, m), 1.78 (2H, m), 1.51 (2H, m). MP: 171° C.

### Example 4

### Preparation of Open-Chain Sulfonamide-nitrile 2Bc

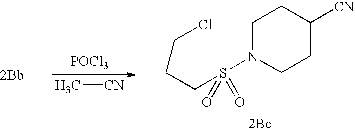

With reference to Scheme IIB (non-alternative method) compound 2Bb prepared in Example 3 (5.0 g, 18.6 mmol), acetonitrile (20 mL), and phosphorous oxychloride (4.28 g, 27.9 mmol) were charged to a 100 mL flask, and heated to 60° C. The reaction was kept at 60° C. for about four hours, and then cooled to 20 to 25° C. The reaction was quenched by the slow addition of 10% aqueous sodium citrate. After quenching, the pH of the reaction was adjusted to 4-5 by the addition of 4 M sodium hydroxide solution. The resulting mixture was extracted with ethyl acetate (2×75 mL), and the combined organic layers were washed with water (25 mL) and brine (25 mL). The solvent was then removed under reduced pressure to yield 4.09 g (87.7%) compound 2Bb as an off-white solid.

¹H NMR (DMSO, 400 MHz): δ 3.73 (2H, dd, J =6.4, 6.4Hz), 3.34 (2H, m), 3.20-3.01 (5H, m), 2.10 (2H, m), 1.94 (2H, m), 1.76 (2H, m). MP=78.8° C.

### Example 5

### Preparation of Sulfonamide-nitrile A2a from 2Bc

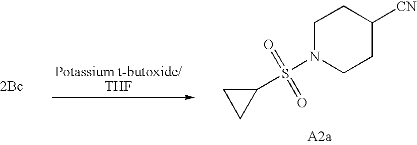

With reference to Step 3 of Scheme IIB (above) potassium tert-butoxide (2.19 g, 17.9 mmol) was dissolved in THF (30 mL) and cooled to −15° C. Compound 2Bc was separately dissolved in tetrahydrofuran (20 mL) and charged to an addition funnel. Compound 2Bc in tetrahydrofuran was then added to the solution of potassium tert-butoxide over about 10 minutes, keeping the temperature below 0° C. The reaction was allowed to stir at −15° C. for about 1 to 2 hours, and was quenched with 10% aqueous citric acid (50 mL). The mixture was extracted with ethyl acetate (60 mL), and the organic layer was washed with water (10 mL), and brine (10 mL). The solvent was removed under reduced pressure to yield crude compound A2a, which was then washed with MTBE (3×15 mL) to yield 2.01 g (78.2%) of compound A2a. The ¹H spectrum was identical to that of compound A2a formed in example 2.

### Example 6

### Preparation of Compound A3 from A2a

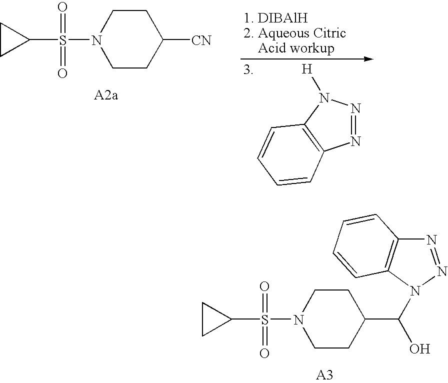

With reference to Scheme 1B1, above, Compound A2a (107 g, 500 mmol) and tetrahydrofuran (640 mL) were charged to a 3 liter jacketed flask and stirred at 20° C. until solids had all dissolved. The reaction was then cooled to about −10° C. Diisobutyl aluminum hydride (DiBAL-H, 400 mL of a 1.5 M solution in toluene, 600 mmol) was then added, keeping the reaction mixture between −15° C. and 0° C. The reaction was stirred at this temperature range for 2 hours after addition of DiBAL-H, and was then quenched into a 25% aqueous solution of citric acid (500 mL), keeping the temperature of the quench below 40° C. The quenched batch was allowed to stir for 1 hour at 20° C., and was then settled and split. The lower, aqueous layer was extracted with toluene (600 mL), the organic layers were pooled, and washed with water (200 mL):

In a separate vessel, benzotriazole (59.6 g, 500 mmol) was dissolved in tetrahydrofuran (500 mL). The benzotriazole solution was then charged to the batch, and the batch was heated to reflux. The batch was then distilled under atmospheric pressure to about 1.1 L (10× volume with respect to A2a) and cooled to about 60° C. Toluene (500 mL) was then added, and the batch was redistilled under atmospheric pressure to 1.1 L. The batch was then cooled to 20-30° C., at which time solid A3 crystallized out. Toluene (500 mL) was then added and the batch was filtered, washed with toluene (500 mL), and dried under vacuum to yield 145 g (86.4%) compound A3 as a white solid.

¹H NMR (DMSO, 400 MHz. n.b. Compound A3 slowly decomposes back to its parent aldehyde and benzotriazole over time when stored in DMSO solution): 8.06 (1H, d, J=8.8 Hz), 7.96 (1H, d, J=8.4 Hz), 7.51 (1H, ddd, J=8.0, 8.0, 0.8 Hz), 7.45-7.40 (2H, m), 6.08 (1H, dd, J=9.2, 6.4 Hz), 3.70 (1H, bd, J=12.0 Hz), 3.48 (1H, bd, J=12.0 Hz), 2.87 (1H, ddd, J=12.8, 12.8, 2.4 Hz), 2.68 (1H, ddd, J=12.0, 12.0, 2.4 Hz), 2.56-2.49 (2H, m), 2.43 (1H, ddddd, J=8.4, 8.4, 8.4, 3.6, 3.6 Hz), 2.14 (1H, bd, J=12.4 Hz), 1.50 (1H, dddd, J=12.4, 12.4, 12.4, 4.4 Hz), 1.25, (1H, dddd, J=12.4, 12.4, 12.4, 4.0 Hz), 0.98-0.85 (5H, m). MP: 139° C.

### Example 7

### Preparation of Compound A4 from A3

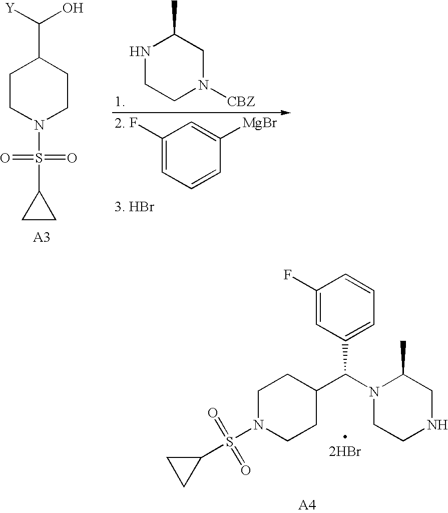

With reference to Scheme 1c, above, compound A3 prepared in Example 6 (20.0 g, 59.5 mmol), 3-(S)-methyl piperazine-1-carboxylic acid benzyl ester (14.6 g, 62.9 mmol), p-toluenesulfonic acid (0.12 g) and toluene (240 ml) were placed into a 1 L, three-necked flask equipped with a Dean-Stark water trap, a mechanical stirrer and a thermometer. The mixture was heated at reflux for 18 hours. The mixture was then distilled to reduce the volume to 10× (200 mL). The mixture was cooled to 0° C.-5° C. and to this was added a solution of 3-fluorophenyl magnesium bromide (1M in THF, 64 ml). A solution of sodium citrate in water (17.6 g/80 ml) was then added to the mixture and stirred for 2 hours. The reaction mixture was settled and split to remove the lower aqueous layer. The organic layer was washed with water (80 mL). The organic solution was extracted with aqueous HBr (48% aqueous, 60 ml). The aqueous HBr solution was agitated at 80° C.-90° C. for 4 hours. The mixture was cooled to 40° C. and to this was added isopropyl alcohol (300 mL). The mixture was held at 40° C. for 2 hours then cooled to 5° C. and held at 5 ° C. for 18 hours to complete the crystallization. The product was filtered and washed with isopropyl alcohol (200 ml). The wet cake was dried in a vacuum oven at 55° C. for 18 hours to give 21.2 g (74.8%) compound A4 as a white solid.

¹H NMR (D₂O, 400 MHz): δ 7.39 (1H, dd, J−14.4, 8.0 Hz), 7.22-7.12 (3H, m), 4.74-4.60 (2H, overlap with HOD peak), 4.49 (1H, d, J =10.0 Hz), 3.79 (1H, bd, J=12.6 Hz), 3.63 (1H, bd, J=12.0 Hz), 3.55 (1H, bd, J=12.8 Hz), 3.44-3.29 (4H, m), 3.21 (1H, dd, J=12.2, 12.2 Hz), 3.10 (1H, bs), 2.84 (1H, dd, J=12.0, 12.0 Hz), 2.70 (1H, dd, J=12.0, 12.0 Hz), 2.40-2.36 (2H, m), 1.93 (1H, bd, J=12.6 Hz), 1.40-1.29 (1H, m), 1.36 (3H, d, J=6.0 Hz), 1.14-1.06 (2H, m), 0.98-0.90 (4H, m). MP: 200° C.

The above description of the invention is intended to be illustrative and not limiting. Various changes or modifications in the embodiments described herein may occur to those skilled in the art. These changes can be made without departing from the scope or spirit of the invention

1. A process for making the compound of Formula I, 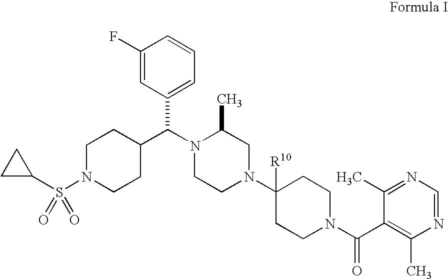
the process comprising:
(a) synthesizing the compound A4 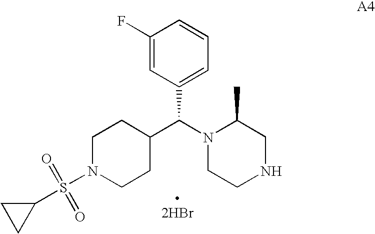
by reacting the compound A3 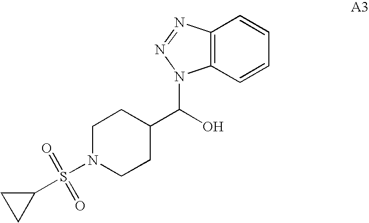
successively with: (i) 4 N-CBZ protected 2 methylpiperidine; (ii) 3-fluorophenyl magnesium bromide; and (iii) HBr in accordance with scheme 1c 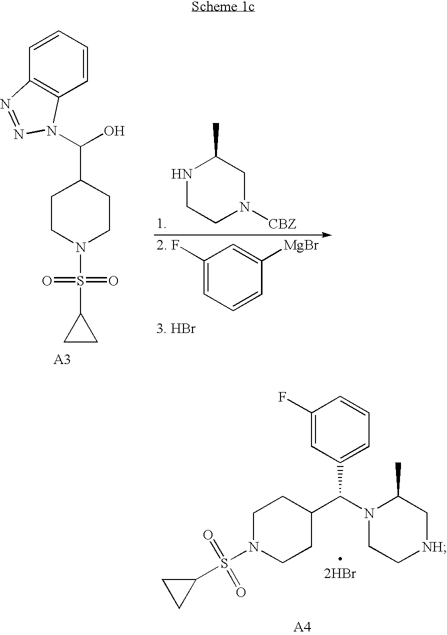
(b) coupling the free base form of intermediate compound A4 formed in Step “a” with the compound of Formula D1 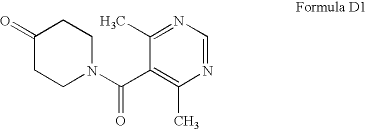
in the presence of a reactant having the form “E-G”, thereby forming the compound of Formula IX, 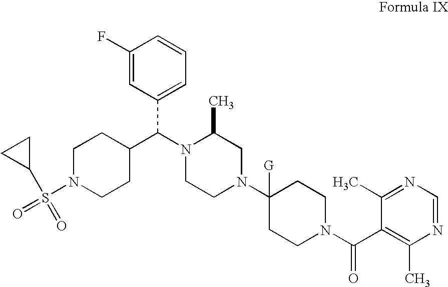
wherein, for the “E-G” reagent, “G” is selected from: (i) ON; (ii) a sulfonate ester of the Formula [—OS(O)₂—R¹], wherein R¹ is selected from an alkyl or aryl group: (iii) halogen; (iv) —C(O)—O—OX₃, wherein “X” is a halogen; and (iv) benzotriazolyl, and wherein “E” is an electrophile capable of scavenging the oxygen of the ketone carbonyl group of the compound of Formula D1 upon nucleophilic attack at the corresponding carbonyl carbon; and
(c) reacting the compound of Formula IX in a suitable solvent with an organometallic reagent supplying an R¹⁰ moiety, where R¹⁰ is selected from an aliphatic and an aromatic moiety, followed by a workup to yield the compound of Formula I.

2. The process of claim 1 wherein said E-G compound is selected from HCN, acetone cyanohydrin; cyclohexanone cyanohydrin; a mixture of (C₂H₅)₂AlCN and Ti(OPr)₄; a mixture of acetic acid and H₂SO₄ admixed with a salt selected from NaHSO₄, KHSO₃ and Na₂S₂O₅ and a cyanide source selected from NaCN and KCN; trimethylsilylcyanide; glycolonitrile; mandelonitrile; glycinonitrile; acetone amino nitrile; and dimethylaminoacetonitrile.

3. The process of claim 2 wherein said organometallic reagent in Step “c” is selected from magenesium, lithium, zinc, and tin organometallic reagents an organometallic reagent supplying an R¹⁰ moiety.

4. The process of claim 3 wherein said R¹⁰ moiety is alkyl, alkenyl, or alkynyl and the E-G compound is acetone cyanohydrin.

5-7. (canceled)

8. The process of claim 1 wherein, in step “a” synthesizing step reaction of scheme 1c: step 1 is carried out in refluxing toluene catalyzed by p-toluenesulfonic acid; step 2 is carried out at a temperature of from about 0 C to about 5 C; and step 3 is carried out by adding aqueous HBr to the reaction mixture.

9. A process for the preparation of a compound of the Formula A3 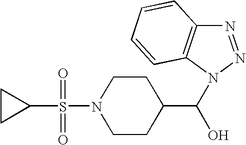
Formula A3
the process comprising:
(a) converting the sulfonamide of Formula A1 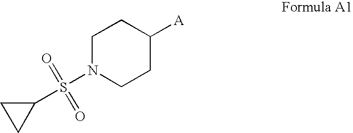
wherein the substituent “A” is selected from: (i) a substituent of the Formula —C(O)—X, wherein “X” is selected from: halogen; trialkylsilane; NR²R³, wherein R² is independently selected from hydrogen, an aliphatic moiety, an aromatic moiety, and a heterocyclic moiety, and R³ is independently selected from hydrogen, O—R², NR²₂, an aliphatic moiety, an aromatic moiety, and a heterocyclic moiety, or R² and R³ taken together form a ring; and —S—R¹ and —O—R¹, wherein R¹ is selected from hydrogen, an aliphatic moiety, including an alkyl, alkyaryl, an aromatic moiety, and a heterocyclic moiety; (ii) an alkyl-alkenyl-substituent of the formula —CHC(R²⁰)₂ wherein R²⁰ is independently selected from H, alkyl and aryl; (iii) —CN; and (iv) —CH₂OH,
to an aldehyde compound of the Formula A2b, 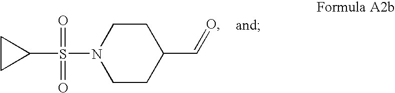
(b) reacting the aldehyde of Formula A2b with benzotriazole to form the benzotriazole-adduct of Formula A3.

10. The process of claim 9 wherein, said “A” substituent on the sulfonamide compound of Formula A1 is CN, and therefore is a compound of Formula A2a and the conversion Step (a) is carried out by reducing the compound of Formula A1 with diisobutyl aluminum hydride (DibAH) followed by an acid workup.

11. The process of claim 10 wherein, the compound of Formula A1 is the cyanide compound of Formula A2a, 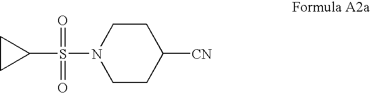
which is provided by treating the compound of Formula A1a, 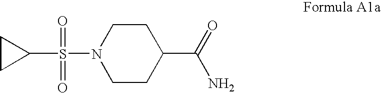
with a dehydrating agent selected from thionyl chloride, phosgene, phosphorous pentoxide and oxalyl chloride.

12. The process of claim 10, wherein, the compound of Formula A1 is the cyanide compound of Formula A2a, 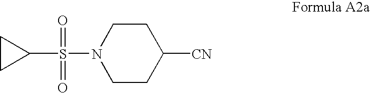
which is provided by treating the compound of Formula A1a, 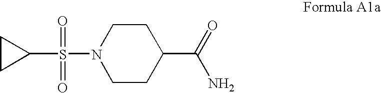
with phosphorous oxychloride.

13. A process for preparing the compound of Formula A1 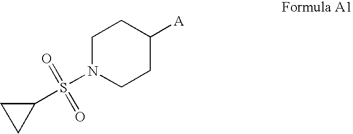
wherein the substituent “A” is selected from CH₂OH, C(O)NH₂, —C(O)OEt, C(O)OH, and derivatives thereof, the process comprising reacting a compound of Formula A1c, 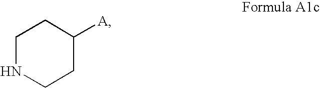
with cyclopropylsulfonyl chloride to form the compound of Formula A1, wherein “A” is as defined above.

14. A process for preparing the compound of Formula A1 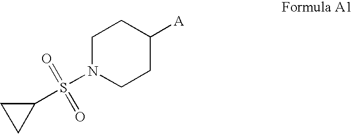
the process comprising:
(a) reacting isonipecotamide (compound 2Ba) with 3-chloropropane-sulfonyl chloride to form the adduct compound 28b in accordance with Step 1; 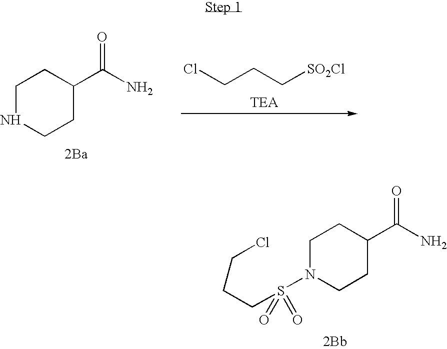
(b) reacting the compound of Formula 2Bb in accordance with a Step 2 scheme selected from Path A, treatment with phosphorous trichloride to provide the corresponding nitrile compound of Formula 2Bc and Path B, treatment with potassium t-butoxide to cyclize the chloropropyl moiety to provide the corresponding chloropropyl sulfonamide compound 2Bd; 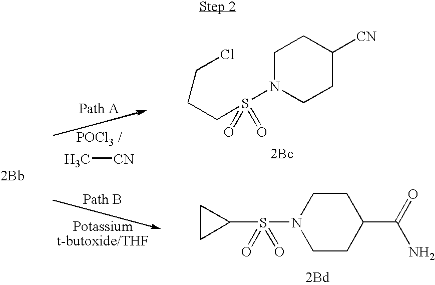
(c) selecting in accordance with Step 3 a reaction from: (i) cyclizing, when Path A is followed in Step 2, the 3-chloropropyl substituent on the sulfonamide substituent of compound 2Bc to form compound A2a; and (ii) converting, when Path B is followed in Step 2, the amide substituent of compound 2Bd to a nitrite substituent, providing the compound of A2a; and 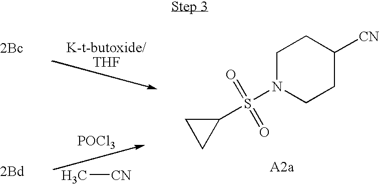
(d) reacting compound A2a with benzotriazole to form benzotriazole adduct compound A3 in accordance with Step 4.

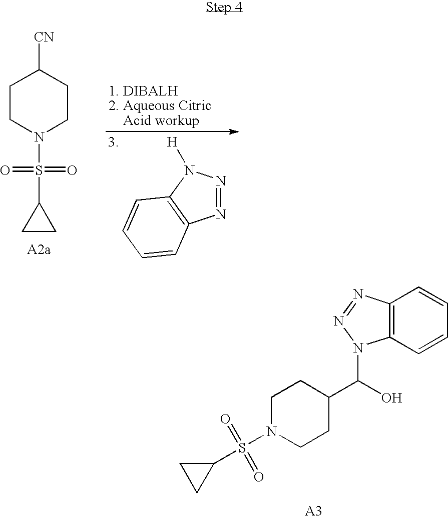

15. A process for providing the compound of Formula A3, 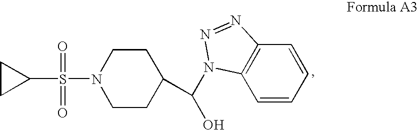
the process comprising:
(a) reacting isonipecotamide of Formula 2Ba, 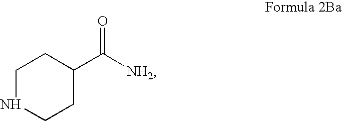
with 3-chloro-n-propane-sulfonyl chloride to form the compound of Formula 2Bb, 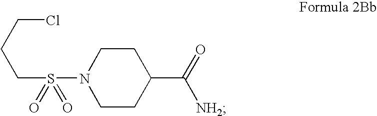
(b) reacting the compound of Formula 2Bb formed in Step (a) with phosphorous oxychloride to form the compound of Formula 2Bc 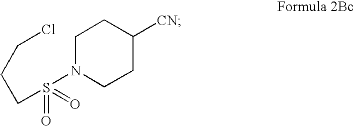
(c) cyclizing the 3-chloro-propane substituent on the compound of Formula 2Bc formed in Step (b) by treating it with a metal alkoxide base to form the compound of Formula A2a, 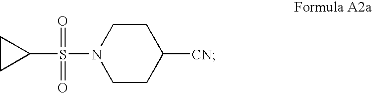
(d) forming an aldehyde of the Formula A2b by reducing the compound of Formula A2a prepared in Step (c), followed by an acid workup to provide the aldehyde compound of Formula A2b, 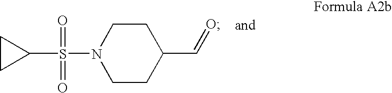
(e) reacting the compound of Formula A2b formed in Step (d) with benzotriazole to form the compound of Formula A3.

16. The process of claim 15 wherein sulfonamide formation Step (a) is carried out in methyl tertiary butyl ether (MTBE) solvent and is catalyzed by the presence of triethylamine (TEA) as a base.

17. The process of claim 16 wherein Step (b), nitrite formation, is carried out in acetonitrile solvent and wherein cyclopropanation step (c) is carried out by treating compound 2Bc with potassium tert-butoxide in tetrahydrofuran (THF) solvent.

18. (canceled)

19. A process for preparing the compound of Formula Id, 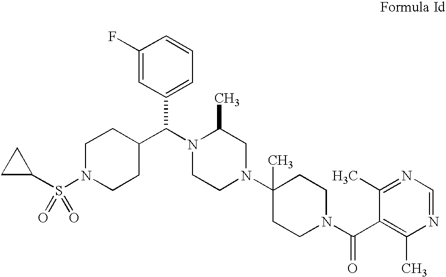
the process comprising:
(i) reacting isonipecotamide with 3-chloropropane-sulfonyl chloride to form the adduct compound 2Bb, 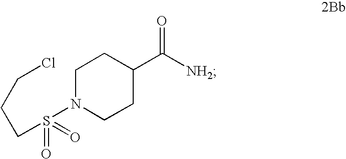
(ii) converting the amide substituent of compound 28b to a nitrile substituent, thereby forming compound 2Bc, 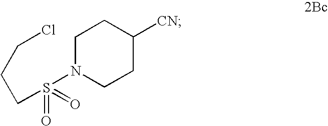
(iii) cyclizing the 3-chloropropyl substituent on the sulfonamide substituent of compound 2Bc to form compound A2a, 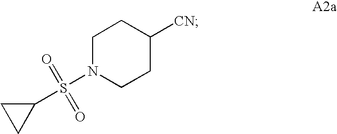
(iv) converting compound A2a to the corresponding aldehyde by reduction of the cyano-substitution with DibAlH followed by an acid workup and subsequently reacting the aldehyde in situ with benzotriazole to form benzotriazole adduct compound of Formula A3, 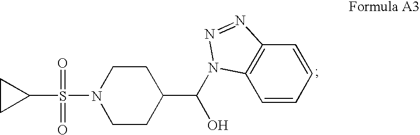
(v) synthesizing the compound of Formula A4. 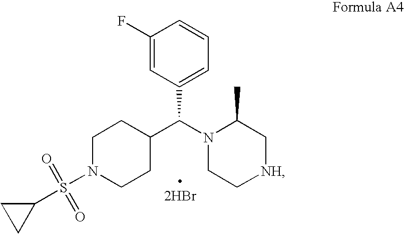
by reacting the compound of Formula A3 formed in Step (iii) successively with: (a) 4 N-CBZ protected 2 methylpiperidine; (b) 3-fluorophenyl magnesium bromide; and (c) HBr;
(vi) liberating the free base of compound A4 from the hydrobromide salt prepared in Step “iv”:
(vii) reacting the free base liberated in Step “v” with the compound of Formula D1, 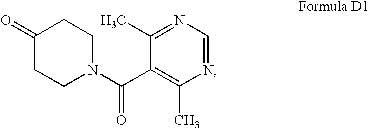
in the presence of a moiety of the Formula “E-G”, where “E” is an electrophile capable of scavenging the oxygen of the ketone carbonyl group of the compound of Formula D1 upon nucleophilic attack at the corresponding carbonyl carbon and “G” is a leaving group selected from the group consisting of ON, halogen, —OSO₂—R¹ (wherein R¹ is selected from an alkyl or aryl group), —C(O)OCX₃ (wherein “X” is a halogen), and benzotriazolyl, to form the compound of Formula IX, 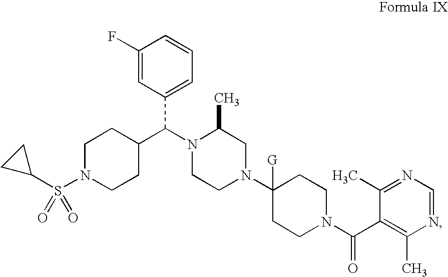
wherein “G” is selected from ON, Z, sulfonate ester [R¹S(O)₃] wherein R¹ is selected from an alkyl or aryl group, halogen, and —(O)C—O—CX₃, wherein “X” is a halogen; and
(viii) reacting the compound of Formula IX formed in Step (vi) with a methyl Grignard reagent in a suitable solvent, followed by a workup, to yield the compound of Formula Id.

20. A process for providing the compound of Formula A3, 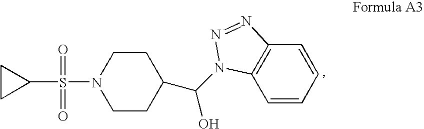
the process comprising:
(a) reacting isonipecotamide of Formula 2Ba, 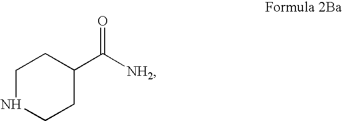
with 3-chloro-n-propane-sulfonyl chloride to form the compound of Formula 2Bb, 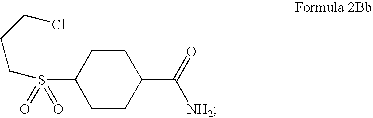
(b) reacting the compound of Formula 2Bb formed in Step (a) with a dehydrating agent selected from thionyl chloride, phosgene, phosphorous pentoxide, and oxalyl chloride to form the compound of Formula 2Bc 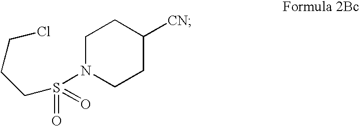
(c) cyclizing the 3-chloro-propane substituent on the compound of Formula 2Bc formed in Step (b) by treating it with a base to form the compound of Formula A2a, 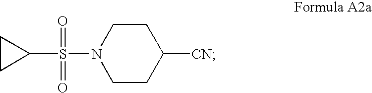
(d) forming an aldehyde of the Formula A2b by reducing the compound of Formula A2a prepared in Step (c), followed by an acid workup to provide the aldehyde compound of Formula A2b, 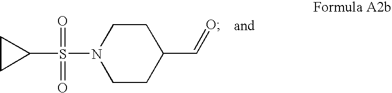
(e) reacting the compound of Formula A2b formed in Step (d) with benzotriazole to form the compound of Formula A3.

21. A process for making the compound of Formula I, 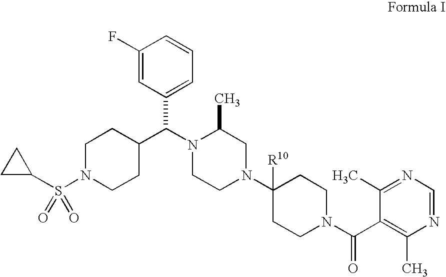
the process comprising:
(a) synthesizing the compound A4 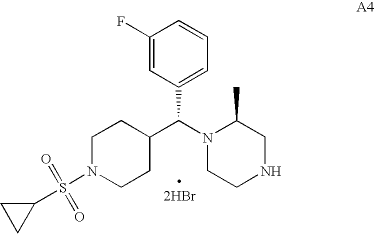
by reacting the compound A3 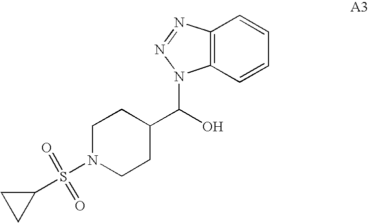
successively with: (i) 4 N-protected 2 methylpiperidine; (ii) 3-fluorophenyl magnesium bromide; and (iii) removing the protecting group “PG” from the 4-N moiety by treatment with an acid selected from HCl, HI, H₂SO₄, AlX₃, wherein “X” is a halogen, BX₃, wherein “X” is a halogen or by hydrogenation using hydrogen gas in the presence of a hydrogenation catalyst, in accordance with scheme 1c 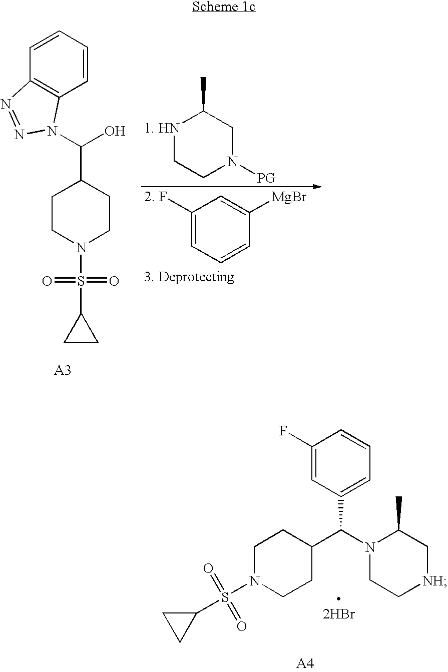
wherein “PG” is an acid-labile or hydrogen reduction removable nitrogen protecting group;
(b) coupling the free base form of intermediate compound A4 formed in Step “a” with the compound of Formula D1 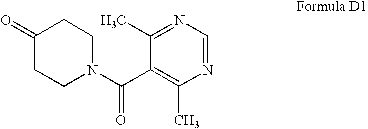
in the presence of a reactant having the form “E-G”, thereby forming the compound of Formula IX, 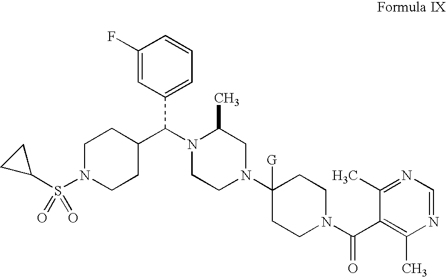
wherein, for the “E-G” reagent, “G” is selected from: (i) CN: (ii) a sulfonate ester of the Formula [—OS(O)₂—R¹], wherein R¹ is selected from an alkyl or aryl group: (iii) halogen; (iv) —C(O)—O—CX₃, wherein “X” is a halogen; and (iv) benzotriazolyl, and wherein “E” is an electrophile capable of scavenging the oxygen of the ketone carbonyl group of the compound of Formula D1 upon nucleophilic attack at the corresponding carbonyl carbon; and
(c) reacting the compound of Formula IX in a suitable solvent with an organometallic reagent supplying an R¹⁰ moiety, where R¹⁰ is selected from an aliphatic and an aromatic moiety, followed by a workup to yield the compound of Formula I.

22. The compounds: 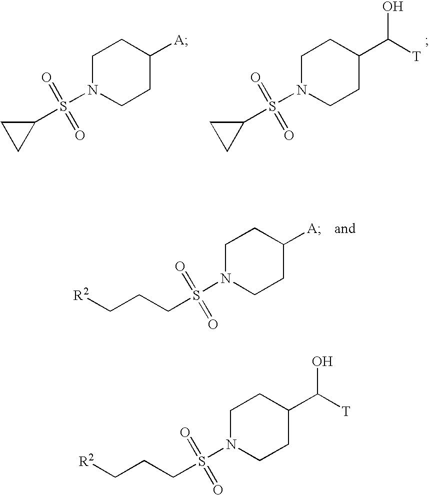
wherein, “A” is as defined above, T is selected from: (i) CN; (ii) a sulfonate ester of the Formula [—OS(O)₂—R¹¹], wherein R¹¹ is selected from an alkyl or aryl group; (iii) halogen; (iv) —C(O)—O—CX₃, wherein “X” is a halogen; (v) triazole; and (vi) benzotriazole, and R^{z} is selected from: a halogen and or O—R¹³ where R¹³ is selected from —C(O)R¹⁴, —C(O)OR¹⁴, or S(O)₂R¹⁴, where R¹⁴ is H, an aliphatic moiety and an aromatic moiety.
